(19) United States
(12) Patent Application Publication
(10) Pub. No.: US 2024/0350818 A1
(43) Pub. Date: Oct. 24, 2024

(54) Implantable Cardioverter-Defibrillator

(71) Applicant: BIOTRONIK SE & Co. KG, Berlin (DE)

(72) Inventors: Thomas DOERR, Berlin (DE); Ingo WEISS, Berlin (DE)

(73) Assignee: BIOTRONIK SE & Co. KG, Berlin (DE)

(21) Appl. No.: 18/685,272

(22) PCT Filed: Aug. 17, 2022
(86) PCT No.: PCT/EP2022/072970

§ 371 Date: Feb. 21, 2024

(30) Foreign Application Priority Data
Sep. 2, 2021 (EP) 21194446.7

(51) Int. Cl. A61N 1/39

(52) U.S. Cl. CPC A61N 1/3956

(57) ABSTRACT

An implantable cardioverter-defibrillator having a housing comprising a processor, a memory unit, a single electrode connection port, a stimulation unit configured to provide an electrode being connected to the electrode connection port with an electric pulse to stimulate a human or animal heart, and a detection unit configured to receive an electric signal of the same heart from the same electrode. According to an aspect of the invention, the electrode connection port is configured to receive a transvenously implantable electrode or a substernally implantable electrode, wherein the memory unit comprises a computer-readable program that causes the processor to operate the stimulation unit and/or the detection unit in a first operational mode if a transvenously implantable electrode is connected to the electrode connection port and in a second operational mode if a substernally implantable electrode is connected to the electrode connection port.

### CROSS-REFERENCE TO RELATED APPLICATIONS

This application is the United States National Phase under 35 U.S.C. § 371 of PCT International Patent Application No. PCT/EP2022/072970, filed on Aug. 17, 2022, which claims the benefit of European Patent Application No. 21194446.7, filed on Sep. 2, 2021, the disclosures of which are hereby incorporated by reference herein in their entireties.

### TECHNICAL FIELD

The present invention relates to an implantable cardioverter-defibrillator according to the preamble of claim 1 and to a defibrillation arrangement comprising such an implantable cardioverter-defibrillator according to the preamble of claim 6.

An implantable cardioverter-defibrillator (ICD) is implanted into the body of a patient and is able to perform a cardioversion by defibrillation and optionally also a pacing of the heart. It is generally possible to connect different kinds of electrodes to an ICD and to program the ICD accordingly in order to work properly with the chosen electrode.

### BACKGROUND

U.S. Pat. No. 5,411,528 A describes an electrically programmable polarity connector for an implantable body tissue stimulator, such as an ICD. In this context, it is described that a physician may program into the ICD's memory the lead type (e.g., transvenous, epicardial patch, subcutaneous, etc.) and the placement of the lead (e.g., right or left ventricle, atrial, superior vena cava, coronary sinus, etc.). This US patent further describes the possibility that any attending physician can interrogate the ICD and determine the exact shocking configuration and, if necessary, proceed to evaluate the effectiveness of the electrode polarity and alter it accordingly.

U.S. Pat. No. 5,441,518 A describes an implantable multi chamber cardioversion and defibrillation system with multiple independently controllable and programmable switched electrode discharge pathways. This independently controlled switching arrangement provides for control over the polarity, phase, direction and timing of all cardioversion and defibrillation countershocks, and allows for the varying of subsequent countershocks after an initial countershock. The switching arrangement is, preferably, both programmable prior to implantation of the system and re-programmable after implantation of the system.

U.S. Publication No. 2004/0215240 A1 describes a reconfigurable cardiac device including a housing, wherein detection circuitry and energy delivery circuitry are provided in the housing. One or more subcutaneous, non-intrathoracic electrodes are coupled to the energy delivery and detection circuitry. A lead interface is provided on the housing and coupled to the energy delivery and detection circuitry. The lead interface is configured to receive at least one lead that includes one or more intrathoracic lead electrodes. A controller is provided in the housing and coupled to the lead interface and the energy delivery and detection circuitry. The system is operable in a first configuration using the subcutaneous electrodes in the absence of the lead and operable in a second configuration using at least one or more of the lead electrodes. The system is capable of providing cardiac activity sensing and stimulation in each of the first and second system configurations, respectively.

The present disclosure is directed toward overcoming one or more of the above-mentioned problems, though not necessarily limited to embodiments that do.

### SUMMARY

It is an object of the present invention to provide an implantable cardioverter-defibrillator that can be used universally for various applications and that offers a higher level of user-friendliness and safety than cardioverter-defibrillators known from prior art.

At least this object is achieved with an implantable cardioverter-defibrillator having the features of claim 1. Such an implantable cardioverter-defibrillator (ICD) has a housing that comprises a processor, a memory unit, exactly one electrode connection port, a stimulation unit, and a detection unit. The stimulation unit is configured to provide an electrode that is connected to the electrode connection port with an electric pulse to stimulate a human or animal heart. The detection unit is designed to receive an electric signal of the same heart with the help of the same electrode. Due to the single electrode connection port, it is only possible to connect one electrode at a time with the ICD.

According to an aspect of the present invention, the electrode connection port is configured to receive either a transvenously implantable electrode or a substernally implantable electrode. Furthermore, the memory unit comprises a computer-readable program that causes the processor to operate the stimulation unit and/or the detection unit in a first operational mode if a transvenously implantable electrode is connected to the electrode connection port and to operate the stimulation unit and/or the detection unit in a second operational mode if a substernally implantable electrode is connected to the electrode connection port.

Thus, the presently claimed ICD automatically detects the kind of electrode that is connected to the electrode connection port so that the correct electrode configuration can be automatically chosen and applied by the ICD. Consequently, a physician can no longer inadvertent incorrectly program the ICD. Furthermore, the claimed ICD only provides one connection port for a single electrode. Thus, it is no longer possible that inadvertently an incorrect connection port for an electrode is chosen. While there exist ICDs on the market that determine the configuration of a connected electrode according to the connection port into which the electrode is inserted, it is apparent that such possibility is a source of errors in case that an electrode is inserted into an electrode connection port which is not intended for this kind of electrode. To circumvent this source of errors, different adapters or electrode connectors are used to avoid the choice of an incorrect electrode connection port. However, this increases the number of necessary parts and the overall complexity of the ICD systems known from prior art.

The presently claimed ICD does not require any adapter. Rather, any transvenously implantable electrode and any substernally implantable electrode having a defined or standardized connection can be connected to the electrode connection port.

The presently claimed ICD can thus be used together with a transvenously implantable electrode or a substernally implantable electrode without making a choice of the electrode prior to implantation. This reduces the logistic effort for physicians that implant the ICD into a patient. Regardless of the number of patients requiring a transvenously implantable electrode or a substernally implantable electrode, the physicians can make use of a bigger pool of ICDs, the application of which is not limited to a specific kind of electrode to be connected to the ICD.

In an embodiment, the stimulation unit is able to apply a desired cardiac therapy to the human or animal heart of the patient carrying the ICD. Appropriate therapies are a shock therapy, an anti-tachycardic pacing (ATP) or a regular pacing (anti-bradycardic pacing) therapy.

In an embodiment, the first operational mode comprises a first set of parameters and algorithms for generating an electric pulse by the stimulation unit and for sensing an electric signal by the detection unit. Likewise, the second operational mode comprises a second set of parameters and algorithms for generating an electric pulse by the stimulation unit and for sensing an electric signal by the detection unit. In this context, the second set differs from the first set. Thus, the necessary parameters and algorithms for detection and stimulation are specifically adapted to the kind of electrode that is connected to the electrode port of the ICD.

In an embodiment, data on the selected parameters and algorithms is transferred to the programming device and can be displayed to a user so as to enable an individual control of the selected parameters and algorithms. Furthermore, the programming device may enable an adaptation of individual parameters and algorithms of the chosen set of parameters and algorithms.

In an embodiment, the first set of parameters and algorithms and the second set of parameters and algorithms comprise a shock energy of the electric pulse to be generated by the stimulation unit. In case of a substernally implanted electrode, the necessary shock energy is regularly higher than in case of a transvenously implanted electrode.

In an embodiment, the first set of parameters and algorithms and the second set of parameters and algorithms also comprise an information on the shock path to be taken by any electric shocks to be delivered by the stimulation unit. Like in case of the shock energy, also the shock path is significantly different when using an intravenously implanted electrode than in case of a substernally implanted electrode.

In an embodiment, the first set of parameters and algorithms and the second set of parameters and algorithms comprise a parameter controlling an activation or a deactivation of a pacing function of the ICD. In case of a connected substernally implanted electrode, no such pacing function is typically necessary. In contrast, if a transvenously implanted electrode is used in connection with the ICD, the ICD can also fulfil according pacing functions of the heart to be stimulated.

In an embodiment, the first operational mode (i.e., the operational mode chosen when a transvenously implantable electrode is connected to the electrode connection port of the housing of the ICD) comprises a safety arrangement or safety system. This safety arrangement prevents the stimulation unit from generating an electric pulse that has voltage and/or an energy being too high. To be more precise, the safety arrangement prevents the generation of a pulse having a voltage and/or an energy exceeding a predetermined threshold.

In an embodiment, the threshold is 60 J or lies in a range of from 40 J to 60 J, in particular of from 45 J to 55 J, in particular around 50 J. Such a threshold is chosen if the energy of the generated pulse is to be limited by the safety arrangement of the first operational mode.

In an embodiment, the threshold is 1000 V or lies in a range of from between 500 V to 1000 V, in particular of from 600 V to 900 V, in particular of from 700 V to 800 V. Such a threshold is chosen if the voltage of the generated pulse is to be limited by the safety arrangement of the first operational mode.

Any combination of the before-mentioned voltages and energies is possible and encompassed from embodiments of the present invention.

In an embodiment, the ICD is able to generate electric pulses having an energy of more than 20 J in order to deliver shock pulses having a sufficiently high shock energy for cardioversion and/or defibrillation. Depending on the type of connected electrode, delivered shock energies lying in a range of from 10 J to 60 J, in particular of from 30 J to 45 J, or in a range of from 20 J to 120 J, in particular of from 30 J to 100 J, in particular of from 40 J to 90 J, in particular of from 50 J to 80 J, in particular of from 60 J to 110 J, in particular of from 70 J to 100 J, in particular of from 80 J to 90 J are particularly appropriate.

In an embodiment, the housing has a volume being smaller than 70 cm³, in particular lying in a range of from 20 cm³ to 70 cm³, in particular of from 30 cm³ to 65 cm³, in particular of from 40 cm³ to 60 cm³, in particular of from 50 cm³ to 55 cm³.

In an embodiment, the housing has a thickness not exceeding 13 mm, in particular lying in a range of from 5 mm to 13 mm, in particular of from 6 mm to 12 mm, in particular of from 7 mm to 11 mm, in particular of from 8 mm to 10 mm.

In an embodiment, the housing has rounded edges having a radius being at least section-wise bigger than 1 mm, in particular lying in a range of from 1 mm to 5 mm, in particular of from 1.5 mm to 4.5 mm, in particular of from 2 mm to 4 mm, in particular of from 2.5 mm to 3.5 mm.

In an embodiment, the ICD has the capability for transmitting data to a home monitoring system to allow an easy monitoring of the proper functioning of the ICD.

In an embodiment, the ICD can be subcutaneously implanted. It can then be denoted as sICD.

In an aspect, the present invention relates to a defibrillation arrangement comprising an implantable cardioverter-defibrillator according to any of the preceding explanations and an electrode connected to the electrode connection port of this implantable cardioverter-defibrillator. As explained above, the electrode is either a transvenously implantable electrode or a substernally implantable electrode. All electrodes that can be connected to the electrode connection port of the ICD comprise-regardless of the specific kind of electrode-the same connector type. Particularly appropriate connectors are IS-1, DF-1, IS4, and DF4 connectors. These types of connectors are standardized and commonly available connectors so that the ICD is connectable to a plurality of widely available electrodes.

In an embodiment, the memory unit of the ICD comprises a computer-readable program that causes the processor to perform the steps explained in the following when executed on the processor.

First, the detection unit and the electrode are used to measure at least one physiologic parameter of a patient to whom the defibrillation arrangement is implanted.

Afterwards, the at least one physiologic parameter is used to determine whether the connected electrode is transvenously implanted or substernally implanted.

Finally, the implantable cardioverter-defibrillator is automatically operated in the first operational mode if the electrode was identified to be a transvenously implanted electrode. Likewise, the ICD is operated in the second operational mode if the electrode was identified to be a substernally implanted electrode. Due to this automatic detection of the type of electrode of the already implanted electrode, a particularly safe and reliable operation of the ICD and its connected electrode is made possible.

In an embodiment, the at least one physiologic parameter is chosen from the group consisting of an impedance and an electrocardiogram. The impedance between an intravenously implanted electrode and the housing of the ICD is significantly higher than the impedance between a substernally implanted electrode and the housing of the ICD.

Therefore, the impedance is a particularly reliable measure for determining whether the electrode connected to the electrode connection port of the ICD is an intravenously implanted electrode or a substernally implanted electrode.

Another reliable measure for determining whether the electrode is a substernally implanted electrode or an intravenously implanted electrode is the evaluation of an electrocardiogram recorded with the electrode, since the signals present in the electrocardiogram differ depending on the location of acquisition of the electrocardiogram.

In an embodiment, the at least one physiologic parameter is an electrocardiogram and the determination whether the connected electrode is a transvenously implanted electrode or a substernally implanted electrode comprises an analysis of a temporal occurrence of signals detected in the electrocardiogram. Such a timing analysis of the electrocardiogram can also reveal whether the connected electrode is transvenously implanted or substernally implanted.

In an embodiment, the at least one physiologic parameter is an electrocardiogram and the step of determining comprises a morphologic analysis of signals detected in the electrocardiogram. Besides the temporal occurrence of signals, also the morphology of the signals in the electrocardiogram differs depending on the location of acquisition of the electrocardiogram.

In an embodiment, the computer-readable program causes the processor to read out an electronic identifier of the electrode. This electronic identifier comprises information as to whether the connected electrode is a transvenously implanted electrode or a substernally implanted electrode. Thus, not only a physiologic parameter of the patient can be used to make a distinction between a transvenously implanted electrode and a substernally implanted electrode, but also such an electronic identifier being present on or in the implanted electrode. An appropriate electronic identifier is an identifier working on the basis of radio-frequency identification (RFID). When using RFID, the electrode comprises, in an embodiment, a transponder, whereas the ICD serves as read out device.

In an embodiment, the defibrillation arrangement is configured to deliver an electric pulse having a voltage of at least 60 V between an electrode pole and a pole of the implantable cardioverter-defibrillator in the first operational mode and in the second operational mode. An electric pulse having such a voltage can typically be denoted as high voltage pulse and is particularly appropriate to achieve cardioversion/defibrillation. In an embodiment, the voltage of such electric pulse lies in a range of from 60 V to 1000 V, in particular of from 80 V to 900 V, in particular of from 100 V to 800 V, in particular of from 200 V to 700 V. in particular of from 300 V to 600 V, in particular of from 400 V to 500 V.

In an embodiment, the connection port comprises a plurality of connector poles. In this context, a first connection configuration between the connector poles and electrode poles of the electrode in the first operational mode differs from a second connection configuration between the connector poles and the electrode poles in the second operational mode. As a result, it is possible to control the electrode poles of the connected electrode in a distinct way depending on the chosen operational mode.

In an embodiment, the connection port comprises four connector poles that provide—in the sequence from the most proximal connector pole to the most distal connector pole—low voltage, low voltage, high voltage, and high voltage. In this context, the term “low voltage” refers to voltages lying in a range of from 0.1 V to less than 60 V (e.g., 59.9 V), in particular of from 1 V to 55 V, in particular of from 5 V to 50 V, in particular of from 10 V to 40 V, in particular of from 20 V to 30 V. Alternatively or additionally, the term “high voltage” refers to voltages lying in a range of from 60 V to 1000 V, in particular of from 80 V to 900 V, in particular of from 100 V to 800 V, in particular of from 200 V to 700 V, in particular of from 300 V to 600 V, in particular of from 400 V to 500 V.

In an embodiment, the first connection configuration establishes an electric contact between the first connector pole (most proximal connector pole) and the tip electrode pole, between the second connector pole (distal and adjacent to the first connector pole) and a ring electrode pole, as well as between the fourth connector pole (most distal connector pole) and a shock coil electrode pole. In this electric connection configuration, the third connector pole (lying between the second connector pole and the fourth connector pole) is not used.

In an embodiment, the second connection configuration establishes an electric contact between the first connector pole (most proximal connector pole) and a sensing electrode pole, between the second connector pole (distal and adjacent to the first connector pole) and another sensing electrode pole, between the third connector pole (distal and adjacent to the second connector pole as well as proximal and adjacent to the fourth connector pole) and a shock coil electrode pole, and between the fourth connector pole (most distal connector pole) and also the shock coil electrode pole. In an embodiment, both lines connected to the shock coil supply electric energy to different ends of the shock coil in each case.

In an embodiment, the defibrillation arrangement is compatible with methods employing magnetic resonance imaging (MRI compatible). This facilitates future examinations of the patient to whom the defibrillation arrangement is implanted. This ameliorates future diagnostic and therapeutic examinations of the patient.

In an aspect, the present invention relates to a first method of operating a defibrillation arrangement according to the preceding explanations. As laid out before, such defibrillation arrangement comprises an implantable cardioverter-defibrillator (ICD) having a housing comprising a processor, a memory unit, and a single electrode connection port. The defibrillation arrangement further comprises an electrode connected to the electrode connection port. The housing of the ICD further comprises a stimulation unit and a detection unit. The stimulation unit serves for providing the electrode with an electric pulse to stimulate a human or animal heart. The detection unit serves for receiving an electric signal of the same heart with the help of the electrode. The method comprises the steps explained in the following.

First, the detection unit and the electrode are used for measuring at least one physiologic parameter of a patient to whom the defibrillation arrangement has been implanted.

Afterwards, the at least one physiologic parameter is used for determining whether the connected electrode is transvenously implanted or substernally implanted.

Finally, the ICD is operated in a first operational mode if the electrode is transvenously implanted and in a second operational mode if the electrode is substernally implanted. Thus, this method enables an automatic and reliable detection of the kind of used electrode and ensures due to the automatic configuration of the operational mode of the ICD a safe and reliable operation of the ICD depending on the connected electrode type.

In an aspect, the present invention relates to a second method of operating a defibrillation arrangement according to the preceding explanations. This method comprises the steps explained in the following.

First, an electronic identifier on or in the electrode is read out by the ICD.

Afterwards, the result of the read-out of the identifier is used for determining whether the connected electrode is transvenously implanted or substernally implanted.

Finally, the ICD is operated in a first operational mode if the electrode is transvenously implanted and in a second operational mode if the electrode is substernally implanted. Thus, also this method enables an automatic and reliable detection of the kind of used electrode and ensures due to the automatic configuration of the operational mode of the ICD a safe and reliable operation of the ICD depending on the connected electrode type. It works independent on a physiologic parameter of the patient, but requires an electrode comprising an electronic identifier.

All embodiments of the implantable cardioverter-defibrillator can be combined in any desired way and can be transferred either individually or in any arbitrary combination to the defibrillation arrangement and to the methods. Likewise, all embodiments of the defibrillation arrangement can be combined in any desired way and can be transferred either individually or in any arbitrary combination to the implantable cardioverter-defibrillator and to the described methods. Finally, all embodiments of the methods can be combined in any desired way and can be transferred either individually or in any arbitrary combination to the described implantable cardioverter-defibrillator, to the defibrillation arrangement, and to the respective other method.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Further details of aspects of the present invention will be explained in the following making reference to exemplary embodiments and accompanying Figures. In the Figures:

FIG. 1A shows a schematic depiction of a first embodiment of a defibrillation arrangement;

FIG. 1B shows a schematic depiction of a second embodiment of a defibrillation arrangement;

FIG. 2A shows a first embodiment of a connection configuration; and

FIG. 2B shows a second embodiment of a connection configuration.

### DETAILED DESCRIPTION

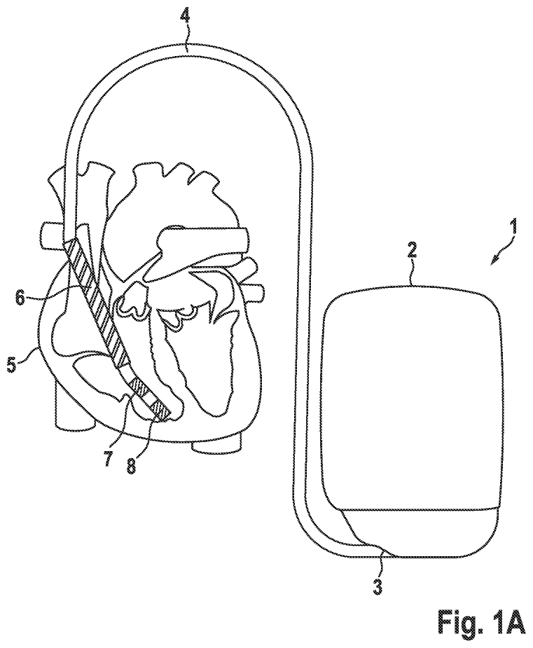

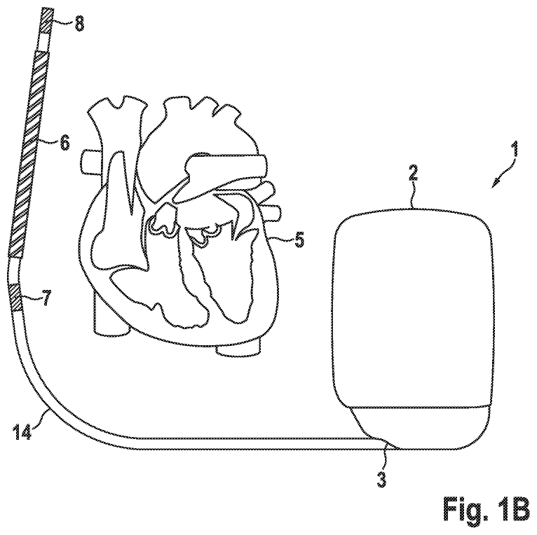

FIG. 1A shows an implantable cardioverter-defibrillator (ICD) 1 having a housing 2 and an electrode connection port 3. A transvenously implanted electrode 4 is connected to the electrode connection port 3. The transvenously implanted electrode 4 is implanted into a human heart 5 via the superior vena cava. The electrode 4 comprises a shock coil 6, a ring electrode pole 7 and a tip electrode pole 8.

By measuring an impedance between the ring electrode 7 or the tip electrode 8 on the one hand and the housing 2 and the other hand, the ICD 1 determines whether the electrode 4 is indeed a transvenously implanted electrode. If the impedance would be too low, a substernally implanted electrode was rather be connected to the electrode connection port 3. Such a situation is illustrated in FIG. 1B. In this and all following Figures, similar elements will be denoted with the same numeral references.

In FIG. 1B, a substernally implanted electrode 14 is connected to the connection port 3 of the housing 2 of the ICD I already shown in FIG. 1A. The substernally implanted electrode 14 is located diagonally above the human heart 5. It also comprises a shock coil 6, a ring electrode 7 and a tip electrode 8. While the ring electrode 7 is located between the shock coil 6 and the tip electrode 8 in case of the transvenously implanted electrode 4 (cf. FIG. 1A), the ring electrode 7 is located proximally of the shock coil 6 in case of the substernally implanted electrode 14. However, other electrode pole arrangements would also be possible.

When determining an impedance between the ring electrode 7 of the substernally implanted electrode 14 and the housing 2 of the ICD 1, the resulting value unambiguously identifies that the electrode 14 is indeed a substernally implanted electrode and not a transvenously implanted electrode. Then, the ICD 1 will be put into an operational mode designed and particularly appropriate for such a substernally implanted electrode 14.

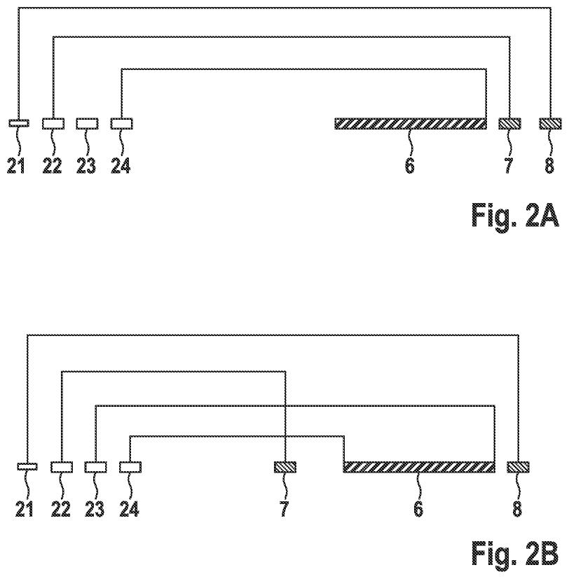

FIG. 2A shows a first connection configuration between the connector poles on the one hand and the electrode poles on the other hand. This first connection configuration is applied in case of a transvenously implanted electrode. A first connector pole 21 is electrically connected with the tip electrode 8 of the transvenously implanted electrode (cf. FIG. 1A for more details). The first connector pole 21 is the most proximal connector pole of all connector poles. A second connector pole 22, which is located adjacent and distally to the first connector pole 21, is connected with the ring electrode 7 of the transvenously implanted electrode. A third connector pole 23 is not connected to any electrode pole. However, a fourth connector pole 24 is connected to one end of the shock coil 6 of the transvenously implanted electrode. Thus, the shock coil 6 comprises a single electric connection so that an electric gradient will be built up when supplying the shock coil 6 with an electric pulse from the fourth connector pole 24. The counter electrode is in each case the housing 2 of the ICD 1 (cf. FIG. 1A for more details).

FIG. 2B shows a second connection configuration that is typically applied when the substernally implanted electrode 14 is connected to the electrode connector 3 of the housing 2 of the ICD 1 (cf. FIG. 1B for more details). Here, the first connector pole 21 (i.e., the most proximal connector pole) is connected to the tip electrode pole 8 of the substernally implanted electrode. Furthermore, the second connector pole 22 is connected to the ring electrode 7. The third connector pole 23, which is located between the second connector pole 22 and the fourth connector pole 24, is connected to a first end of the shock coil 6. The fourth connector pole 24 (most distal connector pole) is connected to the opposite end of the shock coil 6. When applying high-voltage via the third connector pole 23 and the fourth connector pole 24 to the shock coil 6, this voltage is delivered to the shock coil 6 at both ends at the same time. This results in a very homogeneous electric field between the shock coil 6 and the housing 2 of the ICD 1 serving as counter electrode for the shock coil 6 (cf. FIG. 1B for more details).

It is apparent from FIGS. 2A and 2B that the connector poles delivering low voltage to the electrode poles are located adjacent to each other (namely, the first connector pole 21 and the second connector pole 22). Likewise, the two connector poles being able to deliver high voltage to the corresponding electrode poles are also located adjacent to each other (namely, the third connector pole 23 and the fourth connector pole 24).

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. Implantable cardioverter-defibrillator having a housing comprising a processor, a memory unit, a single electrode connection port, a stimulation unit configured to provide an electrode being connected to the electrode connection port with an electric pulse to stimulate a human or animal heart, and a detection unit configured to receive an electric signal of the same heart from the same electrode, wherein
the electrode connection port is configured to receive a transvenously implantable electrode or a substernally implantable electrode, wherein the memory unit comprises a computer-readable program that causes the processor to operate the stimulation unit and/or the detection unit in a first operational mode if a transvenously implantable electrode is connected to the electrode connection port and in a second operational mode if a substernally implantable electrode is connected to the electrode connection port.

2. Implantable cardioverter-defibrillator according to claim 1, wherein the first operational mode comprises a first set of parameters and algorithms for generating an electric pulse by the stimulation unit and for sensing an electric signal by the detection unit and in that the second operational mode comprises a second set of parameters and algorithms for generating an electric pulse by the stimulation unit and for sensing an electric signal by the detection unit, the second set differing from the first set.

3. Implantable cardioverter-defibrillator according to claim 2, wherein the first set of parameters and algorithms and the second set of parameters and algorithms comprise a shock energy of the electric pulse to be generated by the stimulation unit.

4. Implantable cardioverter-defibrillator according to claim 1, wherein the first operational mode comprises a safety arrangement preventing the stimulation unit from generating an electric pulse having a voltage and/or an energy exceeding a predeterminable threshold.

5. Implantable cardioverter-defibrillator according to claim 4, wherein the threshold is chosen from 60 J and 1000 V.

6. Defibrillation arrangement comprising an implantable cardioverter-defibrillator according to claim 1 and an electrode connected to the electrode connection port of the implantable cardioverter-defibrillator.

7. Defibrillation arrangement according to claim 6, wherein the memory unit comprises a computer-readable program that causes the processor to perform the following steps when executed on the processor:
a) measuring, with the detection unit and the electrode, at least one physiologic parameter of a patient to whom the defibrillation arrangement is implanted;
b) determining, based on the at least one physiologic parameter, whether the connected electrode is transvenously implanted or substernally implanted; and
c) operating the implantable cardioverter-defibrillator in the first operational mode if the electrode is transvenously implanted and in the second operational mode if the electrode is substernally implanted.

8. Defibrillation arrangement according to claim 7, wherein the at least one physiologic parameter is chosen from the group consisting of an impedance and an electrocardiogram.

9. Defibrillation arrangement according to claim 7, wherein the at least one physiologic parameter is an electrocardiogram and in that the determining comprises an analysis of a temporal occurrence of signals detected in the electrocardiogram.

10. Defibrillation arrangement according to claim 7, wherein the at least one physiologic parameter is an electrocardiogram and in that the determining comprises a morphologic analysis of signals detected in the electrocardiogram.

11. Defibrillation arrangement according to claim 6, wherein the computer-readable program causes the processor to read out an electronic identifier of the electrode in order to determining whether the connected electrode is a transvenously implanted electrode or a substernally implanted electrode.

12. Defibrillation arrangement according to claim 5, wherein the defibrillation arrangement is configured to deliver an electric pulse having a voltage of at least 60 V between an electrode pole and a pole of the implantable cardioverter-defibrillator both in the first operational mode and in the second operational mode.

13. Defibrillation arrangement according to claim 6, wherein the connection port comprises a plurality of connector poles, wherein a first connection configuration between the connector poles and electrode poles of the connected electrode in the first operational mode differs from a second connection configuration between the connector poles and the electrode poles in the second operational mode.

14. Method for operating a defibrillation arrangement according to claim 6, the defibrillation arrangement comprising i) an implantable cardioverter-defibrillator having a housing comprising a processor, a memory unit, a single electrode connection port, and ii) an electrode connected to the electrode connection port, wherein the housing further comprises a stimulation unit configured to provide the electrode with an electric pulse to stimulate a human or animal heart, and a detection unit configured to receive an electric signal of the same heart from the electrode,
wherein
the method comprises the following steps:
a) measuring, with the detection unit and the electrode, at least one physiologic parameter of a patient to whom the defibrillation arrangement is implanted;
b) determining, based on the at least one physiologic parameter, whether the connected electrode is transvenously implanted or substernally implanted; and
c) operating the implantable cardioverter-defibrillator in a first operational mode if the electrode is transvenously implanted and in a second operational mode if the electrode is substernally implanted.
